(19) United States
(12) Patent Application Publication
(10) Pub. No.: US 2021/0137445 A1
(43) Pub. Date: May 13, 2021

(54) APPARATUS AND METHOD FOR ESTIMATING SKIN BARRIER FUNCTION

(71) Applicant: SAMSUNG ELECTRONICS CO., LTD., Suwon-si (KR)

(72) Inventors: Yun S PARK, Suwon-si (KR); Woochang LEE, Anyang-si (KR); Ho Jun CHANG, Seoul (KR); Eui Seok SHIN, Yongin-si (KR)

(73) Assignee: SAMSUNG ELECTRONICS CO., LTD., Suwon-si (KR)

(21) Appl. No.: 16/850,745

(22) Filed: Apr. 16, 2020

(30) Foreign Application Priority Data
Nov. 12, 2019 (KR) 10-2019-0144336

(51) Int. Cl. A61B 5/00

(52) U.S. Cl. CPC A61B 5/443; A61B 5/0082; A61B 5/0075

(57) ABSTRACT

An apparatus for estimating a skin barrier function or transepidermal water loss of an object may include a spectrum acquisition assembly configured to obtain a Raman spectrum of the object, and a processor configured to extract one or more Type-1 Raman band spectra related to lipids from the obtained Rama spectrum; extract one or more Type-2 Raman band spectra related to lipids and proteins from the obtained Raman spectrum; extract respective features of each of the extracted one or more Type-1 Raman band spectra and the extracted one or more Type-2 Raman band spectra; and estimate the skin barrier function or the transepidermal rater loss of the object based on the extracted features.

### CROSS-REFERENCE TO RELATED APPLICATION(S)

This application claims priority from Korean Patent Application No. 10-2019-0144336, filed on Nov. 12, 2019, in the Korean Intellectual Property Office, the entire disclosure of which is incorporated herein by reference for all purposes.

### BACKGROUND

### 1. Field

Example embodiments consistent with the disclosure relates to technology for estimating a skin barrier function based on Raman spectroscopy.

### 2. Description of Related Art

Human skin, which acts as a waterproof barrier, has many functions including protecting against water loss, preventing bacteria and harmful substances from penetrating into the body, regulating temperature, and the like. These functions of the skin are referred to as skin barrier functions. Transepidermal water loss (referred to as “TEWL”) is used as an indicator of skin barrier function. If the skin barrier functions are faulty, (e.g., if TEWL is high), then allergenic substances may penetrate the skin easily, thereby causing various skin ailments such as atopic dermatitis, and the like. Accordingly, it is particularly important for patients with skin ailments to strengthen and maintain skin barrier function. Furthermore, infants with high transepidermal water loss are more likely to have atopic dermatitis. By proper moisturizing, the prevalence rate of atopic dermatitis in infants may be reduced by half

Therefore, research has been continuously conducted to develop methods for easily and accurately measuring the skin barrier function or transepidermal water loss.

### SUMMARY

According to an aspect of an example embodiment, an apparatus for estimating a skin barrier function or transepidermal water loss of an object may include a spectrum acquisition assembly configured to obtain a Raman spectrum of the object, and a processor configured to extract one or more Type-1 Raman band spectra related to lipids from the obtained Rama spectrum; extract one or more Type-2 Raman band spectra related to lipids and proteins from the obtained Raman spectrum; extract respective features of each of the extracted one or more Type-1 Raman band spectra and the extracted one or more Type-2 Raman band spectra; and estimate the skin barrier function or the transepidermal water loss of the object based on the extracted features.

The spectrum acquisition assembly may be configured to receive the Raman spectrum from an external device.

The spectrum acquisition assembly may be configured to measure the Raman spectrum by emitting light toward the object and receiving Raman scattered light returning from or reflected by the object.

The processor may be configured to extract at least one of Raman band spectra at 1065 cm⁻¹, 1437 cm⁻¹, and 1653 cm⁻¹ as the Type-1 Raman band spectra; and extract Raman band spectra at 2879 cm⁻¹ as the Type-2 Raman band spectra.

The features may include at least one of a peak value and an area value.

The processor may be configured to estimate the skin barrier function by using a skin barrier function estimation model which defines a relationship between the features of the Type-1 Raman band spectra, the features of the Type-2 Raman band spectra, and the skin barrier function; or estimate the transepidermal water loss by using a transepidermal water loss estimation model which defines a relationship between the features of the Type-1 Raman band spectra, the features of the Type-2 Raman band spectra, and the transepidermal water loss.

The skin barrier function estimation model may be generated by regression analysis or machine learning using the features of the Type-1 Raman band spectra, the features of the Type-2 Raman band spectra, and a skin barrier function; and the transepidermal water loss estimation model is generated by regression analysis or machine learning using the features of the Type-1 Raman band spectra, the features of the Type-2 Raman band spectra, and corresponding transepidermal water loss.

The processor may be configured to remove a background signal from the extracted one or more Type-1 Raman band spectra and the extracted one or more Type-2 Raman band spectra, and extract the features of each of the one or more Type-1 Raman band spectra, from which the background signal is removed, and the features of each of the one or more Type-2 Raman band spectra, from which the background signal is removed.

The processor may be configured to generate a baseline by connecting a starting point and an ending point of each of the Type-1 Raman band spectra and the Type-2 Raman band spectra in a straight line or a curved line, and remove the background signal by subtracting the generated baseline from a corresponding Raman band spectrum.

The processor may be configured to remove a background signal from the obtained Raman spectrum, extract the one or more Type-1 Raman band spectra and the one or more Type-2 Raman band spectra from the obtained Raman spectrum, from which the background signal is removed, extract the respective features of each of the extracted one or more Type-1 Raman band spectra and features of each of the extracted one or more Type-2 Raman band spectra, and estimate the skin barrier function or the transepidermal water loss based on the extracted features.

The processor may be configured to estimate a baseline of the obtained Raman spectrum, and remove the background signal by subtracting the estimated baseline from the obtained Raman spectrum.

According to an aspect of an example embodiment, a method of estimating a skin barrier function or transepidermal water loss of an object may include obtaining a Raman spectrum of the object; extracting one or more Type-1 Raman band spectra related to lipids from the obtained Rama spectrum; extracting one or more Type-2 Raman band spectra related to lipids and proteins from the obtained Raman spectrum; removing a background signal from the extracted one or more Type-1 Raman band spectra and the extracted one or more Type-2 Raman band spectra; extracting respective features of each of the one or more Type-1 Raman band spectra, from which the background signal is removed, and each of the one or more Type-2 Raman band spectra, from which the background signal is removed; and estimating the skin barrier function or the transepidermal water loss of the object based on the extracted features.

The obtaining of the Raman spectrum may include receiving the Raman spectrum from an external device.

The obtaining of the Raman spectrum may include measuring the Raman spectrum by emitting light toward the object and receiving Raman scattered light returning from or reflected by the object.

The extracting may include extracting at least one of Raman band spectra at 1065 cm⁻¹, 1437 cm⁻¹, and 1653 cm⁻¹ as the Type-1 Raman band spectra from the obtained Raman spectrum; and extracting Raman band spectra at 2879 cm⁻¹ as the Type-2 Raman band spectra from the obtained Raman spectrum.

The features may include at least one of a peak value and an area value.

The removing of the background signal may include generating a baseline by connecting a starting point and an ending point of each of the Type-1 Raman band spectra and the Type-2 Raman band spectra in a straight line or a curved line, and removing the background signal by subtracting the generated baseline from a corresponding Raman band spectrum.

The estimating of the skin barrier function or the transepidermal water loss may include estimating the skin barrier function by using a skin barrier function estimation model which defines a relationship between the features of the Type-1 Raman band spectra, the features of the Type-2 Raman band spectra, and the skin barrier function; or estimating the transepidermal water loss by using a transepidermal water loss estimation model which defines a relationship between the features of the Type-1 Raman band spectra, the features of the Type-2 Raman band spectra, and the transepidermal water loss.

The skin barrier function estimation model may be generated by regression analysis or machine learning using the features of the Type-1 Raman band spectra, the features of the Type-2 Raman band spectra, and a skin barrier function; and the transepidermal water loss estimation model is generated by regression analysis or machine learning using the features of the Type-1 Raman band spectra, the features of the Type-2 Raman band spectra, and corresponding transepidermal water loss.

According to an aspect of an example embodiment, a method of estimating a skin barrier function or transepidermal water loss of an object may include obtaining a Raman spectrum of an object; removing a background signal from the obtained Raman spectrum; extracting one or more Type-1 Raman band spectra related to lipids from the obtained Raman spectrum, from which the background signal is removed; extracting one or more Type-2 Raman band spectra related to lipids and proteins from the obtained Raman spectrum, from which the background signal is removed; extracting respective features of each of the extracted one or more Type-1 Raman band spectra and the extracted one or more Type-2 Raman band spectra; and estimating the skin barrier function or the transepidermal water loss of the object based on the extracted features

### BRIEF DESCRIPTION OF THE DRAWINGS

The above and/or other aspects of the present disclosure will be more apparent from the following description of example embodiments taken in conjunction with the accompanying drawings, in which:

FIG. 1 is a block diagram illustrating an apparatus for estimating a skin barrier function according to an example embodiment;

FIG. 2 is a block diagram illustrating a spectrum acquisition assembly according to an example embodiment;

FIG. 3 is a block diagram illustrating a processor according to an example embodiment;

FIG. 4 is a diagram explaining a method of extracting Raman band spectra and removing a background signal according to an example embodiment;

FIG. 5 is a block diagram illustrating a processor according to an example embodiment;

FIG. 6 is a block diagram illustrating an apparatus for estimating a skin barrier function according to an example embodiment;

FIG. 7 is a flowchart illustrating a method of estimating a skin barrier function according to an example embodiment; and

FIG. 8 is a flowchart illustrating a method of estimating a skin barrier function according to an example embodiment.

### DETAILED DESCRIPTION

Hereinafter, example embodiments of the present disclosure will be described in detail with reference to the accompanying drawings. It should be noted that wherever possible, the same reference symbols refer to the same parts even in different drawings. In the following description, a detailed description of known functions and configurations incorporated herein will be omitted so as to not obscure the subject matter of the present disclosure.

Process steps described herein may be performed differently from a specified order, unless a specified order is clearly stated in the context of the disclosure. For example, each step may be performed in a specified order, at substantially the same time, in a reverse order, or in a different order.

Further, the terms used throughout the specification are defined in consideration of the functions according to exemplary embodiments, and can be varied according to a purpose of a user or manager, precedent, and the like. Therefore, definitions of the terms should be made on the basis of the overall context of the present disclosure.

It should be understood that, although terms such as “first,” “second,” etc., may be used herein to describe various elements, these elements might not be limited by these terms. These terms might be used to distinguish one element from another. Any references to the singular form of a term may include the plural form of the term unless expressly stated otherwise. In the present specification, it should be understood that terms, such as “including,” “having,” etc., indicate the existence of the features, numbers, steps, actions, components, parts, or combinations thereof, disclosed in the specification, and do not preclude the possibility that one or more other features, numbers, steps, actions, components, parts, or combinations thereof, may exist or may be added.

Expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, all of a, b, and c, or any variations of the aforementioned examples.

Further, components that will be described in the specification are discriminated merely according to functions mainly performed by the components. That is, two or more components can be integrated into a single component. Furthermore, a single component can be separated into two or more components. Moreover, each component can additionally perform some or all of a function executed by another component in addition to the main function thereof. Some or all of the main function of each component can be carried out by another component. Each component may be implemented as hardware, software, or a combination of both.

FIG. 1 is a block diagram illustrating an apparatus for estimating a skin barrier function according to an example embodiment; and FIG. 2 is a block diagram illustrating a spectrum acquisition assembly according to an example embodiment. The apparatus 100 for estimating a skin barrier function is an apparatus for non-invasively estimating a skin barrier function and/or Transepidermal water loss (TEWL) by analyzing a Raman spectrum of an object, and may be mounted in an electronic device. Further, the apparatus 100 for estimating a skin barrier function may be enclosed in a housing to be provided as a separate device. In this case, examples of the electronic device may include a cellular phone, a smartphone, a tablet personal computer (PC), a laptop computer, a personal digital assistant (PDA), a portable multimedia player (PMP), a navigation device, an MP3 player, a digital camera, a wearable device, or the like; and examples of the wearable device may include a wristwatch-type wearable device, a wristband-type wearable device, a ring-type wearable device, a waist belt-type wearable device, a necklace-type wearable device, an ankle band-type wearable device, a thigh band-type wearable device, a forearm band-type wearable device, or the like. However, the electronic device is not limited to the above examples, and the wearable device is neither limited thereto.

Referring to FIG. 1, the apparatus 100 for estimating a skin barrier function includes a spectrum acquisition assembly 110 and a processor 120.

The spectrum acquisition assembly 110 may obtain a Raman spectrum of an object.

In an example embodiment, the spectrum acquisition assembly 110 may obtain a Raman spectrum by receiving the Raman spectrum from an external device which measures and/or stores Raman spectra of the object. In this case, the spectrum acquisition assembly 110 may receive the Raman spectrum from the external device by using various communication techniques such as Bluetooth communication, Bluetooth Low Energy (BLE) communication, Near Field Communication (NFC), wireless local area network (WLAN) communication, Zigbee communication, Infrared Data Association (IrDA) communication, Wireless Fidelity (Wi-Fi) Direct (WFD) communication, Ultra-Wideband (UWB) communication, Ant+communication, Wi-Fi communication, Radio Frequency Identification (RFID) communication, third generation (3G), fourth generation (4G), and fifth generation (5G) telecommunications, and the like, but is not limited thereto.

In another embodiment, the spectrum acquisition assembly 110 may measure a Raman spectrum by emitting light toward the object and receiving Raman scattered light returning from or reflected by the object. To this end, as illustrated in FIG. 2, the spectrum acquisition assembly 110 includes a light source 210, a light collector 220, and a photodetector 230.

The light source 210 may emit light toward the object. For example, the light source 210 may emit light of a predetermined wavelength (e.g., infrared light) toward the object. However, wavelengths of light emitted by the light source 210 may vary based on the purpose of measurement or types of objects to be measured. Further, the light source 210 may be formed of a single light source, or may be formed of an array of a plurality of light sources. If the light source 210 is formed of a plurality of light sources, then the plurality of light sources may emit light of the same wavelength or light of different wavelengths. Further, the plurality of light sources may be classified into a plurality of groups, and each group of the light-sources may emit light of different wavelengths.

In an example embodiment, the light source 210 may include a light emitting diode (LED), a laser diode, and the like, but this is merely an example and is not limited thereto.

Furthermore, the light source 210 may further include a filter (e.g., a long pass filter, a clean up filter, a bandpass filter, etc.) for selecting light of a specific wavelength, and/or an optical element (e.g., a reflecting mirror, etc.) for directing light emitted by the light source 210 toward a desired position of the object.

The light collector 220 may collect Raman scattered light from the object. The light collector 220 may include a filter (e.g., a long pass filter, a clean up filter, etc.), a lens (e.g., a collimating lens, a focusing lens, etc.), a fiber, a waveguide, a grating, and the like.

The photodetector 230 may measure a Raman spectrum by receiving the Raman scattered light collected by the light collector 220. In an embodiment, the photodetector 230 may include a photo diode, a photo transistor (PTr), an image sensor (e.g., a charge-coupled device (CCD), a complementary metal-oxide semiconductor (CMOS), etc.), and the like. The photodetector 230 may be a single device, or may be formed of an array of a plurality of devices.

The processor 120 may process various signals and perform various operations related to estimating a skin barrier function and/or transepidermal water loss of the object. The processor 120 may control the spectrum acquisition assembly 110 at predetermined intervals or in response to a user's request to obtain a Raman spectrum of the object, and may estimate the skin barrier function and/or transepidermal water loss of the object by analyzing the obtained Raman spectrum.

Based on the Raman spectra of the object being obtained, the processor 120 may extract one or more spectra Raman bands related to lipids (hereinafter referred to as “Type-1 Raman band spectra”) and one or more Raman band spectra related to proteins (hereinafter referred to as “Type-2 Raman band spectra”) from the obtained Raman spectra, and the processor 120 may estimate the skin barrier function and/or transepidermal water loss of the object based on the extracted one or more Type-1 Raman band spectra and the extracted one or more Type-2 Raman band spectra. In an embodiment, the processor 120 may extract features from the extracted one or more Type-1 Raman band spectra and the extracted one or more Type-2 Raman band spectra, and may estimate the skin barrier function and/or transepidermal water loss of the object by using the extracted features. In this case, the features may include at least one of a peak value and an area value.

FIG. 3 is a block diagram illustrating a processor according to an example embodiment. The processor 300 of FIG. 3 may be an example of the processor 120 of FIG. 1.

Referring to FIG. 3, the processor 300 includes a spectrum extractor 310, a background signal remover 320, a feature extractor 330, and a skin barrier function estimator 340.

The spectrum extractor 310 may extract one or more Type-1 Raman band spectra and one or more Type-2 Raman band spectra from the Raman spectrum of the object. For example, the spectrum extractor 310 may extract spectra of Raman bands related to lipids as the Type-1 Raman band spectra, and may extract spectra of Raman bands related to both lipids and proteins as the Type-2 Raman band spectra. In this case, information associated with the Raman bands related to lipids and information associated with the Raman bands related to both lipids and proteins may be pre-obtained experimentally, and may be stored in an internal or external memory.

For example, in the Raman spectrum, lipids are related to Raman bands at 1065 cm⁻¹, 1437 cm⁻¹, 1653 cm⁻¹, 2879 cm⁻¹, and the like; and protein is related to Raman bands at 2879 cm⁻¹, and the like. Accordingly, the spectrum extractor 310 may extract Raman band spectra at 1065 cm⁻¹, 1437 cm⁻¹, and 1653 cm⁻¹ as the Type-1 Raman band spectra, and may extract Raman band spectra at 2879 cm⁻¹ as the Type-2 Raman band spectra. In this case, the Raman band may indicate an interval where a peak of a corresponding wave number is formed, and the Raman band spectrum may indicate a spectrum of the corresponding interval. For example, in the case where a peak of a 1065 cm⁻¹ Raman band is formed over a range of 640 cm⁻¹ to 1075 cm⁻¹, the 1065 cm⁻¹ Raman band may be in a range of 640 cm⁻¹ to 1075 cm⁻¹, and spectra of the 1065 cm⁻¹ Raman band may be spectra in an interval of 640 cm⁻¹ to 1075 cm⁻¹.

The background signal remover 320 may remove a background signal, such as fluorescence, and the like, from the extracted one or more Type-1 Raman band spectra and the extracted one or more Type-2 Raman band spectra. In an embodiment, the background signal remover 320 may generate a background line by connecting a starting point and an ending point of each of the extracted Raman band spectra (Type-1 Raman band spectra and Type-2 Raman band spectra) in a straight line or a curved line, and may remove a background signal by subtracting the generated background line from a corresponding Raman band spectrum.

The feature extractor 330 may extract features from each of the Raman band spectra, from which the background signal is removed. In this case, the features may include at least one of a peak value and an area value. For example, the feature extractor 330 may extract a peak value of each Raman band spectrum as a feature from each of the extracted Raman band spectra, from which the background signal is removed, or may calculate an area value of each Raman band spectrum by integrating each of the extracted Raman band spectra, from which the background signal is removed, and may extract the calculated area value of each Raman band spectrum as a feature.

The skin barrier function estimator 340 may estimate a skin barrier function and/or transepidermal water loss of the object based on the extracted features of each Raman band spectrum. For example, the skin barrier function estimator 340 may estimate a skin barrier function and/or transepidermal water loss of the object by using features of the one or more Type-1 Raman band spectra, from which the background signal is removed, features of the one or more Type-2 Raman band spectra, from which the background signal is removed, a skin barrier function estimation model, and a transepidermal water loss estimation model. In this case, the skin barrier function estimation model may define a relationship between the features of the one or more Type-1 Raman band spectra and the one or more Type-2 Raman band spectra, from which the background signal is removed, and a skin barrier function corresponding thereto; and the transepidermal water loss estimation model may define a relationship between the features of the one or more Type-1 Raman band spectra and the one or more Type-2 Raman band spectra, from which the background signal is removed, and transepidermal water loss corresponding thereto. The skin barrier function estimation model and the transepidermal water loss estimation model may be stored in an internal or external memory of the processor 300. In an embodiment, the skin barrier function estimation model and the transepidermal water loss estimation model may be generated by regression analysis or machine learning using the one or more Type-1 Raman band spectra and the one or more Type-2 Raman band spectra, from which the background signal is removed, and the skin barrier function and transepidermal water loss corresponding thereto. In this case, a regression analysis algorithm may include linear regression and non-linear regression, and examples of a machine learning algorithm may include an artificial neural network (ANN), a decision tree, a genetic algorithm, genetic programming, a K-nearest neighbors (k-NN) algorithm, a radial basis function (RBF) network , a random forest, a support vector machine (SVM), a deep-learning algorithm, and the like.

In an embodiment, the skin barrier function estimation model and the transepidermal water loss estimation model may be represented by the following Equation 1.

G=a₁x₁+ . . . +aₙxₙ+b₁y₁+ . . . +bₘyₘ+a₀ [Equation 1]

Referring to Equation 1, G denotes the skin barrier function or the transepidermal water loss; xᵢ (i=1, . . . , and n) denotes the features of the Type-1 Raman band spectra, from which the background signal is removed; yⱼ (j=1, . . . , and m) denotes the Type-2 Raman band spectra, from which the background signal is removed; and a₀, aᵢ (i=1, . . . , and n), and bⱼ (j=1, . . . , and m) denote coefficients. In this case, a₀, aᵢ (i=1, . . . , and n), and bⱼ (j=1, . . . , and m) may be calculated using a regression analysis algorithm.

FIG. 4 is a diagram explaining a method of extracting Raman band spectra and removing a background signal according to an example embodiment.

Referring to FIGS. 3 and 4, by analyzing a Raman spectrum 410 of the object, the spectrum extractor 310 may determine that a peak at 1437 cm⁻¹, which is related to a lipid, is formed over a range of 1395 cm⁻¹ to 1470 cm⁻¹, and may extract a spectrum 420 over a range of 1395 cm⁻¹ to 1470 cm⁻¹ as a Raman band spectrum related to a lipid. In this case, the spectrum 420 of 1395 cm⁻¹ to 1470 cm⁻¹ may be a spectrum at wave numbers, at which a first-order differential value of a Raman spectrum, obtained by removing a background signal such as fluorescence, and the like, from the Raman spectrum 410, starts to change from a negative number to a positive number.

The background signal remover 320 may generate a baseline 430 by connecting a starting point and an ending point of the extracted Raman band spectrum 420 in a straight line or a curved line, and may generate a Raman band spectrum 440, from which a background signal such as fluorescence, and the like, is removed, by subtracting the baseline 430 from the extracted Raman band spectrum 420.

In addition, the feature extractor 330 may extract features of the Raman band spectrum 440 from the Raman band spectrum 440, from which the background signal is removed.

FIG. 5 is a block diagram illustrating a processor according to an example embodiment. The processor 500 of FIG. 5 may be another example of the processor 120 of FIG. 1.

Referring to FIG. 5, the processor 500 includes a background signal remover 510, a spectrum extractor 520, a feature extractor 530, and a skin barrier function estimator 540.

The background signal remover 510 may remove a background signal, such as fluorescence, and the like, from a Raman spectrum of an object. That is, the background signal remover 510 may remove a background signal from the entire Raman spectrum. In an embodiment, the background signal remover 510 may estimate a baseline of the Raman spectrum, and may remove the background signal by subtracting the estimated baseline from the Raman spectrum. In this case, the baseline may be estimated using a first-order differential method, a rolling-ball method, and the like. The first-order differential method uses characteristics of background noise exhibiting a gradual change over the entire range. In the first-order differential method, a baseline is estimated by differentiating a spectrum, finding a significant peak, cutting out the corresponding peak area, and performing interpolation. Further, in the rolling-ball method, a trace of a highest point of a hypothetical ball that rolls underneath a spectrum is considered as a baseline.

The spectrum extractor 520 may extract one or more Type-1 Raman band spectra and one or more Type-2 Raman band spectra from the Raman spectrum, from which the background signal is removed. For example, the spectrum extractor 520 may extract spectra of Raman bands, related to lipids, as the Type-1 Raman band spectra, and may extract spectra of Raman bands, related to both lipids and proteins, as the Type-2 Raman band spectra. In this case, information associated with the Raman bands related to lipids and information associated with the Raman bands related to both lipids and proteins may be pre-obtained experimentally, and may be stored in an internal or external memory.

The feature extractor 530 may extract features of each of the extracted Raman band spectra. In this case, the features may include at least one of a peak value and an area value. For example, the feature extractor 530 may extract a peak value of each Raman band spectrum as a feature from each of the extracted Raman band spectra, or may calculate an area value of each Raman band spectrum by integrating each of the extracted Raman band spectra, and may extract the calculated area value of each Raman band spectrum as a feature

The skin barrier function estimator 540 may estimate a skin barrier function and/or transepidermal water loss of the object based on the extracted features of each Raman band spectrum. In this case, the skin barrier function estimator 540 may estimate the skin barrier function and/or transepidermal water loss of the object by using a skin barrier function estimation model and a transepidermal water loss estimation model, which are represented by Equation 1 as shown above elsewhere herein.

FIG. 6 is a block diagram illustrating an apparatus for estimating a skin barrier function according to an example embodiment.

The apparatus 600 for estimating a skin barrier function is an apparatus for non-invasively estimating a skin barrier function and/or transepidermal water loss by analyzing a Raman spectrum of an object, and may be mounted in an electronic device. Further, the apparatus 600 for estimating a skin barrier function may be enclosed in a housing to be provided as a separate device. In this case, examples of the electronic device may include a cellular phone, a smartphone, a tablet PC, a laptop computer, a PDA, a PMP, a navigation device, an MP3 player, a digital camera, a wearable device, and the like; and examples of the wearable device may include a wristwatch-type wearable device, a wristband-type wearable device, a ring-type wearable device, a waist belt-type wearable device, a necklace-type wearable device, an ankle band-type wearable device, a thigh band-type wearable device, a forearm band-type wearable device, or the like. However, the electronic device is not limited to the above examples, and the wearable device is neither limited thereto.

Referring to FIG. 6, the apparatus 600 for estimating a skin barrier function includes the spectrum acquisition assembly 110, the processor 120, an input interface 610, a storage 620, a communication interface 630, and an output interface 640. Here, the spectrum acquisition assembly 110 and the processor 120 are described above with reference to FIGS. 1 to 5, such that detailed description thereof will be omitted.

The input interface 610 may receive input of various operation signals from a user. In an embodiment, the input interface 610 may include a keypad, a dome switch, a touch pad (e.g., a static pressure touch pad, a capacitive touch pad, or the like), a jog wheel, a jog switch, a hardware (H/W) button, and the like. Particularly, the touch pad, which forms a layer structure with a display, may be referred to as a touch screen.

The storage 620 may store programs or commands for operation of the apparatus 600 for estimating a skin barrier function, and may store data input to and output from the apparatus 600 for estimating a skin barrier function. Further, the storage 620 may store Raman spectra, a skin barrier function estimation model, a transepidermal water loss estimation model, an estimated skin barrier function value, an estimated transepidermal water loss value, and the like. The storage 620 may include at least one storage medium of a flash type memory, a hard disk type memory, a multimedia card micro type memory, a card type memory (e.g., a secure digital (SD) memory, an extreme digital (XD) memory, etc.), a random access memory (RAM), a static RAM (SRAM), a read only memory (ROM), an electrically erasable programmable ROM (EEPROM), a programmable ROM (PROM), a magnetic memory, a magnetic disk, and an optical disk, and the like. Further, the apparatus 600 for estimating a skin barrier function may communicate with an external storage medium, such as web storage, and the like, which performs a storage function of the storage 620 via the Internet.

The communication interface 630 may communicate with an external device. For example, the communication interface 630 may transmit, to the external device, the data input to and stored in the apparatus 600 for estimating a skin barrier function, data processed by the apparatus 600 for estimating a skin barrier function, and the like; or may receive, from the external device, various data for estimating a skin barrier function and/or transepidermal water loss.

In this case, the external device may be medical equipment using the data input to and stored in the apparatus 600 for estimating a skin barrier function, the data processed by the apparatus 600 for estimating a skin barrier function, and the like, a printer to print out results, or a display to display the results. In addition, the external device may be a digital television (TV), a desktop computer, a cellular phone, a smartphone, a tablet PC, a laptop computer, a PDA, a PMP, a navigation device, an MP3 player, a digital camera, a wearable device, or the like, but the external device is not limited thereto.

The communication interface 630 may communicate with the external device by using one or more of Bluetooth communication, BLE communication, NFC, WLAN communication, Zigbee communication, IrDA communication, WFD communication, UWB communication, Ant+ communication, Wi-Fi communication, RFID communication, 3G, 4G, and 5G telecommunications, and the like. However, this is merely an example and not intended to be limiting.

The output interface 640 may output the data input to and stored in the apparatus 600 for estimating a skin barrier function, the data processed by the apparatus 600 for estimating a skin barrier function, and the like. In an embodiment, the output interface 640 may output the data input to and stored in the apparatus 600 for estimating a skin barrier function, the data processed by the apparatus 600 for estimating a skin barrier function, and the like, by using at least one of an acoustic method, a visual method, and a tactile method. The output interface 640 may include a display, a speaker, a vibrator, and the like.

FIG. 7 is a flowchart illustrating a method of estimating a skin barrier function according to an example embodiment. The method of estimating a skin barrier function of FIG. 7 may be performed by the apparatuses 100 and 600 for estimating a skin barrier function.

Referring to FIG. 7, the apparatus for estimating a skin barrier function may obtain a Raman spectrum of an object in operation 710. For example, the apparatus for estimating a skin barrier function may obtain a Raman spectrum by receiving the Raman spectrum from an external device which measures and/or stores Raman spectra of the object, or may measure a Raman spectrum by emitting light toward the object and receiving Raman scattered light returning from or reflected by the object.

The apparatus for estimating a skin barrier function may extract one or more Type-1 Raman band spectra and one or more Type-2 Raman band spectra from the Raman spectrum of the object in operation 720. For example, the apparatus for estimating a skin barrier function may extract spectra of Raman bands related to lipids as the Type-1 Raman band spectra, and may extract spectra of Raman bands related to both lipids and proteins as the Type-2 Raman band spectra. In this case, information associated with the Raman bands related to lipids and information associated with the Raman bands related to both lipids and proteins may be pre-obtained experimentally, and may be stored in an internal or external memory of the apparatus for estimating a skin barrier function.

The apparatus for estimating a skin barrier function may remove a background signal, such as fluorescence, and the like, from the extracted one or more Type-1 Raman band spectra and the extracted one or more Type-2 Raman band spectra in operation 730. In an embodiment_{—} the apparatus for estimating a skin barrier function may generate a background line by connecting a starting point and an ending point of each of the extracted Raman band spectra (Type-1 Raman band spectra and Type-2 Raman band spectra) in a straight line or a curved line, and may remove a background signal by subtracting the generated background line from a corresponding Raman band spectrum.

The apparatus for estimating a skin barrier function may extract features of each of the Raman band spectra, from which the background signal is removed, in operation 740. For example, the apparatus for estimating a skin barrier function may extract a peak value of each Raman band spectrum as a feature from each of the extracted Raman band spectra, from which the background signal is removed, or may calculate an area value of each Raman band spectrum by integrating each of the extracted Raman band spectra, from which the background signal is removed, and may extract the calculated area value of each Raman band spectrum as a feature.

The apparatus for estimating a skin barrier function may estimate a skin barrier function and/or transepidermal water loss of the object based on the extracted features of each Raman band spectrum in operation 750. For example, the apparatus for estimating a skin barrier function may estimate a skin barrier function and/or transepidermal water loss of the object by using features of the one or more Type-1 Raman band spectra, from which the background signal is removed, features of the one or more Type-2 Raman band spectra, from which the background signal is removed, a skin barrier function estimation model, and a transepidermal water loss estimation model. In this case, the skin barrier function estimation model may define a relationship between the features of the one or more Type-1 Raman band spectra and the one or more Type-2 Raman band spectra, from which the background signal is removed, and a skin barrier function corresponding thereto; and the transepidermal water loss estimation model may define a relationship between the features of the one or more Type-1 Raman band spectra and the one or more Type-2 Raman band spectra, from which the background signal is removed, and transepidermal water loss corresponding thereto. The skin barrier function estimation model and the transepidermal water loss estimation model may be represented by Equation 1 as shown above elsewhere herein.

FIG. 8 is a flowchart illustrating a method of estimating a skin barrier function according to an example embodiment. The method of estimating a skin barrier function of FIG. 8 may be performed by the apparatuses 100 and 600 for estimating a skin barrier function.

Referring to FIG. 8, the apparatus for estimating a skin barrier function may obtain a Raman spectrum of an object in operation 810. For example, the apparatus for estimating a skin barrier function may obtain a Raman spectrum by receiving the Raman spectrum from an external device which measures and/or stores Raman spectra of the object, or may measure a Raman spectrum by emitting light toward the object and receiving Raman scattered light returning from or reflected by the object.

The apparatus for estimating a skin barrier function may remove a background signal, such as fluorescence and the like, from a Raman spectrum of an object in operation 820. In an embodiment, the apparatus for estimating a skin barrier function may estimate a baseline of the Raman spectrum, and may remove the background signal by subtracting the estimated baseline from the Raman spectrum. In this case, the baseline may be estimated using a first-order differential method, a rolling-ball method, and the like. Here, the first-order differential method uses characteristics of background noise exhibiting a gradual change over the entire range. In the first-order differential method, a baseline is estimated by differentiating a spectrum, finding a significant peak, cutting out the corresponding peak area, and performing interpolation. Further, in the rolling-ball method, a trace of a highest point of a hypothetical ball that rolls underneath a spectrum is considered as a baseline.

The apparatus for estimating a skin barrier function may extract one or more Type-1 Raman band spectra and one or more Type-2 Raman band spectra from the Raman spectrum, from which the background signal is removed, in operation 830. For example, the apparatus for estimating a skin barrier function may extract spectra of Raman bands, related to lipids, as the Type-1 Raman band spectra, and may extract spectra of Raman bands, related to both lipids and proteins, as the Type-2 Raman band spectra. In this case, information associated with the Raman bands related to lipids and information associated with the Raman bands related to both lipids and proteins may be pre-obtained experimentally, and may be stored in an internal or external memory of the apparatus for estimating a skin barrier function.

The apparatus for estimating a skin barrier function may extract features of each of the extracted Raman band spectra in operation 840. For example, the apparatus for estimating a skin barrier function may extract a peak value of each Raman band spectrum as a feature from each of the extracted Raman band spectra, or may calculate an area value of each Raman band spectrum by integrating each of the extracted Raman band spectra, from which the background signal is removed, and may extract the calculated area value of each Raman band spectrum as a feature.

The apparatus for estimating a skin barrier function may estimate a skin barrier function and/or transepidermal water loss of the object based on the extracted features of each Raman band spectrum in operation 850.

The example embodiments of the present disclosure can be implemented by computer-readable code written on a non-transitory computer-readable medium and executed by a processor. Code and code segments for implementing the example embodiments of the present disclosure can be deduced by computer programmers of ordinary skill in the art. The computer-readable medium may be any type of recording device in which data is stored in a computer-readable manner. Examples of the computer-readable medium include a ROM, a RAM, a CD-ROM, a magnetic tape, a floppy disc, an optical disk, and the like. Further, the computer-readable medium can be distributed over a plurality of computer systems connected to a network so that code is written thereto and executed therefrom in a decentralized manner.

Although example embodiments been described herein, it will be understood by those skilled in the art that various changes and modifications can be made without changing technical ideas and features of the present disclosure. Thus, it is clear that the above-described example embodiments are illustrative in all aspects and are not intended to limit the present disclosure.

What is claimed is:

1. An apparatus for estimating a skin barrier function or transepidermal water loss of an object, the apparatus comprising:
a spectrum acquisition assembly configured to obtain a Raman spectrum of the object; and
a processor configured to:
extract one or more Type-1 Raman band spectra related to lipids from the obtained Rama spectrum;
extract one or more Type-2 Raman band spectra related to lipids and proteins from the obtained Raman spectrum;
extract respective features of each of the extracted one or more Type-1 Raman band spectra and the extracted one or more Type-2 Raman band spectra; and
estimate the skin barrier function or the transepidermal water loss of the object based on the extracted features.

2. The apparatus of claim 1, wherein the spectrum acquisition assembly is further configured to receive the Raman spectrum from an external device.

3. The apparatus of claim 1, wherein the spectrum acquisition assembly is further configured to measure the Raman spectrum by emitting light toward the object and receiving Raman scattered light returning from or reflected by the object.

4. The apparatus of claim 1, wherein the processor is further configured to:
extract at least one of Raman band spectra at 1065 cm⁻¹, 1437 cm⁻¹, and 1653 cm⁻¹ as the Type-1 Raman band spectra; and
extract Raman band spectra at 2879 cm⁻¹ as the Type-2 Raman band spectra.

5. The apparatus of claim 1, wherein the features comprise at least one of a peak value and an area value.

6. The apparatus of claim 1, wherein the processor is further configured to:
estimate the skin barrier function by using a skin barrier function estimation model which defines a relationship between the features of the Type-1 Raman band spectra, the features of the Type-2 Raman band spectra, and the skin barrier function: or
estimate the transepidermal water loss by using a transepidermal water loss estimation model which defines a relationship between the features of the Type-1 Raman band spectra, the features of the Type-2 Raman band spectra, and the transepidermal water loss.

7. The apparatus of claim 6, wherein the skin barrier function estimation model is generated by regression analysis or machine learning using the features of the Type-1 Raman band spectra, the features of the Type-2 Raman band spectra, and corresponding skin barrier function; and
the transepidermal water loss estimation model is generated by regression analysis or machine learning using the features of the Type-1 Raman band spectra, the features of the Type-2 Raman band spectra, and corresponding transepidermal water loss.

8. The apparatus of claim 1, wherein the processor is further configured to:
remove a background signal from the extracted one or more Type-1 Raman band spectra and the extracted one or more Type-2 Raman band spectra, and
wherein the processor is configured to extract the features of each of the one or more Type-1 Raman band spectra, from which the background signal is removed, and the features of each of the one or more Type-2 Raman band spectra, from which the background signal is removed.

9. The apparatus of claim 8, wherein the processor is further configured to:
generate a baseline by connecting a starting point and an ending point of each of the Type-1 Raman band spectra and the Type-2 Raman band spectra in a straight line or a curved line, and
remove the background signal by subtracting the generated baseline from a corresponding Raman band spectrum.

10. The apparatus of claim 1, wherein the processor is further configured to:
remove a background signal from the obtained Raman spectrum,
extract the one or more Type-1 Raman band spectra and the one or more Type-2 Raman band spectra from the obtained Raman spectrum, from which the background signal is removed,
extract the respective features of each of the extracted one or more Type-1 Raman band spectra and features of each of the extracted one or more Type-2 Raman band spectra, and
estimate the skin barrier function or the transepidermal water loss based on the extracted features.

11. The apparatus of claim 10, wherein the processor is further configured to:
estimate a baseline of the obtained Raman spectrum, and
remove the background signal by subtracting the estimated baseline from the obtained Raman spectrum.

12. A method of estimating a skin barrier function or transepidermal water loss of an object, the method comprising:
obtaining a Raman spectrum of the object;
extracting one or more Type-1 Raman band spectra related to lipids from the obtained Rama spectrum;
extracting one or more Type-2 Raman band spectra related to lipids and proteins from the obtained Raman spectrum;
removing a background signal from the extracted one or more Type-1 Raman band spectra and the extracted one or more Type-2 Raman band spectra;
extracting respective features of each of the one or more Type-1 Raman band spectra, from which the background signal is removed, and each of the one or more Type-2 Raman band spectra, from which the background signal is removed; and
estimating the skin barrier function or the transepidermal water loss of the object based on the extracted features.

13. The method of claim 12, wherein the obtaining of the Raman spectrum comprises receiving the Raman spectrum from an external device.

14. The method of claim 12, wherein the obtaining of the Raman spectrum comprises measuring the Raman spectrum by emitting light toward the object and receiving Raman scattered light returning from or reflected by the object.

15. The method of claim 12, wherein the extracting comprises:
extracting at least one of Raman band spectra at 1065 cm⁻¹, 1437 cm⁻¹, and 1653 cm⁻¹ as the Type-1 Raman band spectra from the obtained Raman spectrum; and
extracting Raman band spectra at 2879 cm⁻¹ as the Type-2 Raman band spectra from the obtained Raman spectrum.

16. The method of claim 12, wherein the features include at least one of a peak value and an area value.

17. The method of claim 12, wherein the removing of the background signal comprises generating a baseline by connecting a starting point and an ending point of each of the Type-1 Raman band spectra and the Type-2 Raman band spectra in a straight line or a curved line, and removing the background signal by subtracting the generated baseline from a corresponding Raman band spectrum.

18. The method of claim 12, wherein the estimating of the skin barrier function or the transepidermal water loss comprises:
estimating the skin barrier function by using a skin barrier function estimation model which defines a relationship between the features of the Type-1 Raman band spectra, the features of the Type-2 Raman band spectra, and the skin barrier function; or
estimating the transepidermal water loss by using a transepidermal water loss estimation model which defines a relationship between the features of the Type-1 Raman band spectra, the features of the Type-2 Raman band spectra, and the transepidermal water loss.

19. The method of claim 18, wherein the skin barrier function estimation model is generated by regression analysis or machine learning using the features of the Type-1 Raman band spectra, the features of the Type-2 Raman band spectra, and corresponding skin barrier function; and
the transepidermal water loss estimation model is generated by regression analysis or machine learning using the features of the Type-1 Raman band spectra, the features of the Type-2 Raman band spectra, and corresponding transepidermal water loss.

20. A method of estimating a skin barrier function or transepidermal water loss of an object, the method comprising:
obtaining a Raman spectrum of an object;
removing a background signal from the obtained Raman spectrum;
extracting one or more Type-1 Raman band spectra related to lipids from the obtained Raman spectrum, from which the background signal is removed;
extracting one or more Type-2 Raman band spectra related to lipids and proteins from the obtained Raman spectrum, from which the background signal is removed;
extracting respective features of each of the extracted one or more Type-1 Raman band spectra and the extracted one or more Type-2 Raman band spectra; and
estimating the skin barrier function or the transepidermal water loss of the object based on the extracted features.
